(19) United States
(12) Patent Application Publication
(10) Pub. No.: US 2016/0129118 A1
(43) Pub. Date: May 12, 2016

(54) COHESIVE LIQUID BOLUS COMPRISING BIOACTIVES

(71) Applicant: NESTEC S.A., Vevey (CH)

(72) Inventors: Robert Redgwell, Eden Prairie, MN (US); Adam Burbidge, Arzier (CH); Jan Engmann, Lausanne (CH); Simina Popa Nita, Morgers (CH); Jane Durga, Miex (CH)

(21) Appl. No.: 14/897,293

(22) PCT Filed: Jun. 4, 2014
(86) PCT No.: PCT/EP2014/061592

§ 371 Date: Dec. 10, 2015

(30) Foreign Application Priority Data
Jun. 14, 2013 (EP) 13172153.2

(51) Int. Cl. A61K 47/36; A61K 31/198; A61K 31/19; A61K 35/20; A61K 31/20; A61K 31/202; A61K 31/201; A61K 31/205; A61K 31/14; A61K 31/59; A61K 31/16; A61K 31/05; A61K 31/122; A61K 31/385; A23L 2/52; A61K 9/00

(52) U.S. Cl. CPC A61K 47/36; A61K 9/0095; A61K 31/198; A61K 31/19; A61K 35/20; A61K 31/20; A61K 31/202; A61K 31/201; A61K 31/205; A61K 31/14; A61K 31/59; A61K 31/16; A61K 31/05; A61K 31/122; A61K 31/385; A23L 1/296; A23L 2/52; A23V 2002/00

(57) ABSTRACT

This invention relates to a cohesive thin liquid bolus comprising an aqueous solution of at least one food grade biopolymer and at least one bioactive compound, to the use of said cohesive thin liquid bolus for promoting safer swallowing of food boluses in dysphagic patients and to a method for preparing the bolus.

### FIELD OF THE INVENTION

This invention relates to a cohesive thin liquid bolus comprising an aqueous solution of at least one food grade biopolymer and at least one bioactive compound such as anabolic compounds, anti-catabolic compounds, cell function or neuromuscular junction stimulating compounds and cell energy metabolism stimulating compounds. The invention further relates to the use of said cohesive thin liquid bolus for promoting safer swallowing of food boluses for patients having difficulty in swallowing and to a method for preparing the bolus.

### BACKGROUND OF THE INVENTION

Dysphagia is the medical term for the symptom of difficulty in swallowing. Epidemiological studies estimate a prevalence rate of 16% to 22% among individuals over 50 years of age.

Esophageal dysphagia affects a large number of individuals of all ages, but is generally treatable with medications and is considered a less serious form of dysphagia. Esophageal dysphagia is often a consequence of mucosal, mediastinal, or neuromuscular diseases. Mucosal (intrinsic) diseases narrow the lumen through inflammation, fibrosis, or neoplasia associated with various conditions (e.g., peptic stricture secondary to gastroesophageal reflux disease, esophageal rings and webs [e.g., sideropenic dysphagia or Plummer-Vinson syndrome], esophageal tumors, chemical injury [e.g., caustic ingestion, pill esophagitis, sclerotherapy for varices], radiation injury, infectious esophagitis, and eosinophilic esophagitis). Mediastinal (extrinsic) diseases obstruct the esophagus by direct invasion or through lymph node enlargement associated with various conditions (tumors [e.g., lung cancer, lymphoma], infections [e.g., tuberculosis, histoplasmosis], and cardiovascular [dilated auricula and vascular compression]). Neuromuscular diseases may affect the esophageal smooth muscle and its innervation, disrupting peristalsis or lower esophageal sphincter relaxation, or both, commonly associated with various conditions (achalasia [both idiopathic and associated with Chagas disease], scleroderma, other motility disorders, and a consequence of surgery [i.e., after fundoplication and antireflux interventions]). It is also common for individuals with intraluminal foreign bodies to experience acute esophageal dysphagia.

Oral pharyngeal dysphagia, on the other hand, is a very serious condition and is generally not treatable with medication. Oral pharyngeal dysphagia also affects individuals of all ages, but is more prevalent in older individuals. Worldwide, oral pharyngeal dysphagia affects approximately 22 million people over the age of 50. Oral pharyngeal dysphagia is often a consequence of an acute event, such as a stroke, brain injury, or surgery for oral or throat cancer. In addition, radiotherapy and chemotherapy may weaken the muscles and degrade the nerves associated with the physiology and nervous innervation of the swallow reflex. It is also common for individuals with progressive neuromuscular diseases, such as Parkinson's Disease, to experience increasing difficulty in swallowing initiation. Representative causes of oropharyngeal dysphagia include those associated neurological illnesses (brainstem tumors, head trauma, stroke, cerebral palsy, Guillain-Barre syndrome, Huntington's disease, multiple sclerosis, polio, post-polio syndrome, Tardive dyskinesia, metabolic encephalopathies, amyotrophic lateral sclerosis, Parkinson's disease, dementia), infectious illnesses (diphtheria, botulism, Lyme disease, syphilis, mucositis [herpetic, cytomegalovirus, candida, etc.]), autoimmune illnesses (lupus, scleroderma, Sjogren's syndrome), metabolic illnesses (amyloidosis, cushing's syndrome, thyrotoxicosis, Wilson's disease), myopathic illnesses (connective tissue disease, dermatomyositis, myasthenia gravis, myotonic dystrophy, oculopharyngeal dystrophy, polymyositis, sarcoidosis, paraneoplastic syndromes, inflammatory myopathy), iatrogenic illnesses (medication side effects [e.g., chemotherapy, neuroleptics, etc.], post surgical muscular or neurogenic, radiation therapy, corrosive [pill injury, intentional]), and structural illnesses (cricopharyngeal bar, Zenker's diverticulum, cervical webs, oropharyngeal tumors, osteophytes and skeletal abnormalities, congenital [cleft palate, diverticulae, pouches, etc.]).

Dysphagia is not generally diagnosed although the disease has major consequences on patient health and healthcare costs. Individuals with more severe dysphagia generally experience a sensation of impaired passage of food from the mouth to the stomach, occurring immediately after swallowing. Among community dwelling individuals, perceived symptoms may bring patients to see a doctor. Among institutionalized individuals, health care practitioners may observe symptoms or hear comments from the patient or his/her family member suggestive of swallowing impairment and recommend the patient be evaluated by a specialist. As the general awareness of swallowing impairments is low among front-line practitioners, dysphagia often goes undiagnosed and untreated. Yet, through referral to a swallowing specialist (e.g., speech language pathologist), a patient can be clinically evaluated and dysphagia diagnosis can be determined.

Severity of dysphagia may vary from: (i) minimal (perceived) difficulty in safely swallowing foods and liquids, (ii) an inability to swallow without significant risk for aspiration or choking, and (iii) a complete inability to swallow. Commonly, the inability to properly swallow foods and liquids may be due to food boluses being broken up into smaller fragments, which may enter the airway or leave unwanted residues in the oropharyngeal and/or esophageal tract during the swallowing process (e.g., aspiration). If enough material enters the lungs, it is possible that the patient may drown on the food/liquid that has built up in the lungs. Even small volumes of aspirated food may lead to bronchopneumonia infection, and chronic aspiration may lead to bronchiectasis and may cause some cases of asthma.

“Silent aspiration,” a common condition among elderly, refers to the aspiration of oropharyngeal contents such as secretions, food, or liquid due to a lack of pharyngeal reflex in the absence of cough, throat clearance or distress. People may compensate for less-severe swallowing impairments by self-limiting the diet. The aging process itself, coupled with chronic diseases such as hypertension or osteoarthritis, predisposes elderly to (subclinical) dysphagia that may go undiagnosed and untreated until a clinical complication such as pneumonia, dehydration, malnutrition (and related complications) occurs.

Pneumonia is a common clinical consequence of dysphagia. The condition often requires acute hospitalization and emergency room visits. Among those that develop pneumonia due to aspiration, the differential diagnosis of ‘aspiration pneumonia’ is not necessarily indicated as a result of current care practices. Based on U.S. healthcare utilization surveys from recent years, pneumonia accounted for over one million hospital discharges and an additional 392,000 were attributable to aspiration pneumonia. Individuals who have general pneumonia as the principal diagnosis have a mean 6 day hospital length of stay and incur over $18,000 in costs for hospital care. It is expected that aspiration pneumonia would carry higher costs for hospital care, based on a mean 8 day length of hospital stay. Pneumonia is life threatening among persons with dysphagia, the odds of death within 3 months is about 50% (van der Steen et al. 2002). In addition, an acute insult such as pneumonia often initiates the downward spiral in health among elderly. An insult is associated with poor intakes and inactivity, resulting in malnutrition, functional decline, and frailty. Specific interventions (e.g., to promote oral health, help restore normal swallow, or reinforce a swallow-safe bolus) would benefit persons at risk for (due to aspiration of oropharyngeal contents, including silent aspiration) or experiencing recurrent pneumonia.

Similar to pneumonia, dehydration is a life-threatening clinical complication of dysphagia. Dehydration is a common co-morbidity among hospitalized individuals with neurodegenerative diseases (thus, likely to have a swallowing impairment). The conditions of Alzheimer's disease, Parkinson's disease, and multiple sclerosis account for nearly 400,000 U.S. hospital discharges annually, and up to 15% of these patients suffer dehydration. Dehydration as the principal diagnosis is associated with a mean 4 day length of hospital stay and over $11,000 in costs for hospital care. Nevertheless, dehydration is an avoidable clinical complication of dysphagia.

Malnutrition and related complications (e.g., [urinary tract] infections, pressure ulcers, increased severity of dysphagia [need for more-restricted food options, tube feeding, and/or PEG placement and reduced quality of life], dehydration, functional decline and related consequences [falls, dementia, frailty, loss of mobility, and loss of autonomy]) can arise when swallowing impairment leads to fear of choking on food and liquids, slowed rate of consumption, and self-limited food choices. If uncorrected, inadequate nutritional intake exacerbates dysphagia as the muscles that help facilitate normal swallow weaken as physiological reserves are depleted. Malnutrition is associated with having a more than 3-times greater risk of infection. Infections are common in individuals with neurodegenerative diseases (thus, likely to have a chronic swallowing impairment that jeopardizes dietary adequacy). The conditions of Alzheimer's disease, Parkinson's disease, and multiple sclerosis account for nearly 400,000 U.S. hospital discharges annually, and up to 32% of these patients suffer urinary tract infection.

Moreover, malnutrition has serious implications for patient recovery. Malnourished patients have longer length of hospital stay, are more likely to be re-hospitalized, and have higher costs for hospital care. Malnutrition as the principal diagnosis is associated with a mean 8 day length of hospital stay and nearly $22,000 in costs for hospital care. Furthermore, malnutrition leads to unintentional loss of weight and predominant loss of muscle and strength, ultimately impairing mobility and the ability to care for oneself. With the loss of functionality, caregiver burden becomes generally more severe, necessitating informal caregivers, then formal caregivers, and then institutionalization. However, malnutrition is an avoidable clinical complication of dysphagia.

Among persons with neurodegenerative conditions (e.g., Alzheimer's disease), unintentional weight loss as a marker of malnutrition precedes cognitive decline. In addition, physical activity can help stabilize cognitive health. Thus, it is important to ensure nutritional adequacy among persons with neurodegenerative conditions to help them have the strength and endurance to participate in regular therapeutic exercise and guard against unintentional weight loss, muscle wasting, loss of physical and cognitive functionality, frailty, dementia, and progressive increase in caregiver burden.

The economic costs of dysphagia are associated with hospitalization, re-hospitalization, loss of reimbursement due to pay for performance (“P4P”), infections, rehabilitation, loss of work time, clinic visits, use of pharmaceuticals, labor, care taker time, childcare costs, quality of life, increased need for skilled care. Dysphagia and aspiration impact quality of life, morbidity and mortality. Twelve-month mortality is high (45%) among individuals in institutional care who have dysphagia and aspiration. The economic burden of the clinical consequences arising from lack of diagnosis and early management of dysphagia are significant.

Considering the prevalence of dysphagia, possible complications related thereto, and the costs associated with same, there is still a need for providing an improved method for treating swallowing disorders, which method can minimize the risk of standard bolus therapy, promotes safer swallowing of food boluses and prevents or treats the clinical complications of dysphagia in patients suffering from aspiration. Such a method would improve the lives of a large and growing number of persons with swallowing impairments. Specific interventions (e.g., to promote oral health, help restore normal swallow, or reinforce a swallow-safe bolus) can enable persons to eat orally (vs. being tube fed and/or requiring PEG placement) and experience the psycho-social aspects of food associated with general well being while guarding against the potentially negative consequences that result from lack of adequate swallowing ability. Improvements in the intake of nutrition by dysphagic patients may also enable such patients to swallow a wider variety of food and beverage products safely and comfortably, which may lead to an overall healthier condition of the patient and prevent further health-related decline.

### SUMMARY OF THE INVENTION

These needs are met by the present invention, which provides a novel, nutritionally reinforced, swallow-safe bolus and a method for preparing the same. The bolus of the invention helps to prevent or treat the clinical complications of dysphagia by guarding the patient against unintentional weight loss and muscle wasting, and thus helps to avoid functional decline, loss of physical and cognitive functionality, frailty, and ultimately loss of mobility in patients suffering from a swallowing disorder.

Accordingly, in a first aspect, the invention relates to a bolus selected from a cohesive liquid having (i) a shear viscosity of less than about 400 mPas, and (ii) a relaxation time, determined by a Capillary Breakup Extensional Rheometry (CaBER) experiment, of more than 10 ms (milliseconds) at a temperature of 20° C.; the bolus comprising: (1) an aqueous solution of at least one food grade biopolymer selected from the group consisting of botanical hydrocolloids, microbial hydrocolloids, animal hydrocolloids, algae hydrocolloids and any combination thereof; and (2) at least one bioactive compound selected from: (a) Anabolic compounds; (b) Anti-catabolic compounds; (c) Cell function or neuromuscular junction stimulating compounds; and (d) Cell energy metabolism stimulating compounds.

In a preferred embodiment of the first aspect of the invention, (a) The anabolic compounds are optionally selected from the group consisting of leucine, leucine metabolites, α-hydroxyisocaproic acid (HICA), glutamine, arginine, citrulline, creatine, whey, medium chain fatty acids (MCFA) and combinations thereof; (b) The anti-catabolic compounds are optionally selected from the group consisting of polyunsaturated fatty acids (PUFA), ω-3 fatty acids, carnitine, creatine, and combinations thereof; (c) The cell function or neuromuscular junction stimulating compounds are optionally selected from the group consisting of choline, vitamin D, creatine, Oleoylethanolamide (OEA), resveratrol, and combinations thereof; (d) The cell energy metabolism stimulating compounds are optionally selected from the group consisting of antioxidants, coenzyme Q10, creatine, lipoic acid, carnitine, resveratrol, medium chain fatty acids (MCFA) and combinations thereof.

In a further embodiment of the first aspect of the invention, the bolus is preferably selected from a cohesive thin liquid having (i) a shear viscosity of less than about 50 mPas, preferably from 5 to 45 mPas, more preferably from 10 to 40 mPas, and most preferably from 20 to 30 mPas, when measured at a shear rate of 50 s−1; and (ii) a relaxation time, determined by a Capillary Breakup Extensional Rheometry (CaBER) experiment, of more than 10 ms (milliseconds) at a temperature of 20° C.

In another embodiment of the first aspect of the invention, the bolus is preferably selected from a cohesive thickened liquid having (i) a shear viscosity of more than about 50 mPas, preferably from 55 to 350 mPas, more preferably from 60 to 200 mPas, and most preferably from 70 to 100 mPas, when measured at a shear rate of 50 s−1; and (ii) a relaxation time, determined by a Capillary Breakup Extensional Rheometry (CaBER) experiment, of more than 10 ms (milliseconds) at a temperature of 20° C.

It is further preferred that in the first aspect of the invention the relaxation time is less than about 2000 ms, preferably from about 20 ms to about 1000 ms, more preferably from about 50 ms to about 500 ms, and most preferably from about 100 ms to about 200 ms, at a temperature of 20° C.

In a further preferred embodiment of the first aspect of the invention, the aqueous solution comprises at least one food grade biopolymer in a concentration of from at least 0.01 wt % to 25 wt %, preferably from at least 0.1 wt % to 15 wt %, and most preferably from at least 1 wt % to 10 wt %.

In a yet further preferred embodiment of the first aspect of the invention, the food grade biopolymer is a botanical hydrocolloid selected from plant-extracted gums, plant-derived mucilages, or combinations thereof, wherein, optionally, the plant-extracted gums are selected from the group consisting of okra gum, konjac mannan, tara gum, locust bean gum, guar gum, fenugreek gum, tamarind gum, cassia gum, acacia gum, gum ghatti, pectins, modified celluloses (e.g., carboxymethyl cellulose, methyl cellulose, hydroxylpropyl methyl cellulose, hydroxypropyl cellulose), tragacanth gum, karaya gum, or any combinations thereof, and preferably the plant-extracted gum is okra gum; and/or the plant-derived mucilages are selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, or combinations thereof, and preferably the plant-derived mucilage is kiwi fruit mucilage and/or cactus mucilage.

A further preferred embodiment of the invention relates to the bolus of the above first aspect in administrable form selected from the group consisting of a pharmaceutical formulation, a nutritional formulation, a nutritional supplement, a dietary supplement, a functional food, a beverage product, a full meal, a nutritionally complete formula, and combinations thereof.

A yet further preferred embodiment of the invention relates to the bolus of the above first aspect for use in treating a swallowing disorder, for use in promoting safe swallowing of nutritional products, and/or for use in mitigating the risks of aspiration during swallowing of nutritional products in a patient in need of same.

A particularly preferred embodiment of the invention relates to the bolus of the above first aspect for use in supporting, maintaining and/or improving strength and/or muscle health in a patient suffering from a swallowing disorder.

In a second aspect, the invention relates to a method for making a bolus selected from a cohesive liquid, the method comprising the steps of: (1) Providing an aqueous solution of at least one food grade biopolymer capable of providing to the bolus: (i) a shear viscosity of less than about 400 mPas, and (ii) a relaxation time, determined by a Capillary Breakup Extensional Rheometry (CaBER) experiment, of more than 10 ms (milliseconds) at a temperature of 20° C.; and (2) Adding to said aqueous solution at least one bioactive compound selected from: (a) Anabolic compounds; (b) Anti-catabolic compounds; (c) Cell function or neuromuscular junction stimulating compounds; and (d) Cell energy metabolism stimulating compounds.

In a preferred embodiment of the second aspect of the invention, (a) The anabolic compounds are optionally selected from the group consisting of leucine, leucine metabolites, α-hydroxyisocaproic acid (HICA), glutamine, arginine, citrulline, creatine, whey, medium chain fatty acids (MCFA) and combinations thereof; (b) The anti-catabolic compounds are optionally selected from the group consisting of polyunsaturated fatty acids (PUFA), ω-3 fatty acids, carnitine, creatine, and combinations thereof; (c) The cell function or neuromuscular junction stimulating compounds are optionally selected from the group consisting of choline, vitamin D, creatine, Oleoylethanolamide (OEA), resveratrol, and combinations thereof; (d) The cell energy metabolism stimulating compounds are optionally selected from the group consisting of antioxidants, coenzyme Q10, creatine, lipoic acid, carnitine, resveratrol, medium chain fatty acids (MCFA) and combinations thereof.

In a further embodiment of the method according to the second aspect of the invention, the bolus is preferably selected from a cohesive thin liquid, wherein in step (1) an aqueous solution of at least one food grade biopolymer is provided which is capable of providing to the bolus: (i) a shear viscosity of less than about 50 mPas, preferably from 5 to 45 mPas, more preferably from 10 to 40 mPas, and most preferably from 20 to 30 mPas, when measured at a shear rate of 50 s−1; and (ii) a relaxation time, determined by a Capillary Breakup Extensional Rheometry (CaBER) experiment, of more than 10 ms (milliseconds) at a temperature of 20° C.

In another embodiment of the method according to the second aspect of the invention, the bolus is preferably selected from a cohesive thickened liquid, wherein in step (1) an aqueous solution of at least one food grade biopolymer is provided which is capable of providing to the bolus: (i) a shear viscosity of more than about 50 mPas, preferably from 55 to 350 mPas, more preferably from 60 to 200 mPas, and most preferably from 70 to 100 mPas, when measured at a shear rate of 50 s−1; and (ii) a relaxation time, determined by a Capillary Breakup Extensional Rheometry (CaBER) experiment, of more than 10 ms (milliseconds) at a temperature of 20° C.

It is further preferred that in the second aspect of the invention the relaxation time is less than about 2000 ms, preferably from about 20 ms to about 1000 ms, more preferably from about 50 ms to about 500 ms, and most preferably from about 100 ms to about 200 ms, at a temperature of 20° C.

In a further preferred embodiment of the second aspect of the invention, the aqueous solution comprises the at least one food grade biopolymer in a concentration of from at least 0.01 wt % to 25 wt %, preferably from at least 0.1 wt % to 15 wt %, and most preferably from at least 1 wt % to 10 wt %.

In a yet further preferred embodiment of the second aspect of the invention, the food grade biopolymer is a botanical hydrocolloid selected from plant-extracted gums, plant-derived mucilages, or combinations thereof, wherein, optionally, the plant-extracted gums are selected from the group consisting of okra gum, konjac mannan, tara gum, locust bean gum, guar gum, fenugreek gum, tamarind gum, cassia gum, acacia gum, gum ghatti, pectins, modified celluloses (e.g., carboxymethyl cellulose, methyl cellulose, hydroxylpropyl methyl cellulose, hydroxypropyl cellulose), tragacanth gum, karaya gum, or any combinations thereof, and preferably the plant-extracted gum is okra gum; and/or the plant-derived mucilages are selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, or combinations thereof, and preferably the plant-derived mucilage is kiwi fruit mucilage and/or cactus mucilage.

A further preferred embodiment of the invention relates to the method of the above second aspect further comprising the optional step (3) of diluting the bolus, preferably in an aqueous dilution ranging from 2:1 to 50:1, more preferably from 3:1 to 20:1 and most preferably from 5:1 to 10:1.

A yet further preferred embodiment of the invention relates to the method of the above second aspect further comprising the step (4) of bringing the bolus in an administrable form selected from the group consisting of a pharmaceutical formulation, a nutritional formulation, a nutritional supplement, a dietary supplement, a functional food, a beverage product, a full meal, a nutritionally complete formula, and combinations thereof.

Other aspects and embodiments of the present invention are described below.

### DETAILED DESCRIPTION OF THE INVENTION

The present invention provides a bolus selected from a cohesive liquid having (i) a shear viscosity of less than about 400 mPas, and (ii) a relaxation time, determined by a Capillary Breakup Extensional Rheometry (CaBER) experiment, of more than 10 ms (milliseconds) at a temperature of 20° C.; the bolus comprising: (1) an aqueous solution of at least one food grade biopolymer selected from the group consisting of botanical hydrocolloids, microbial hydrocolloids, animal hydrocolloids, algae hydrocolloids and any combination thereof; and (2) at least one bioactive compound selected from: (a) Anabolic compounds; (b) Anti-catabolic compounds; (c) Cell function or neuromuscular junction stimulating compounds; and (d) Cell energy metabolism stimulating compounds.

### Bolus

As used herein, term “bolus” refers to a physical portion of a food or beverage that can be swallowed by a patient. Said bolus may be in solid, semi-solid or liquid form and may comprise one or more nutrients, foods or nutritional supplements. Preferably, the bolus is a liquid. It is further preferred that the bolus has a volume the patient can consume in one swallowing event. The preferred volume of the liquid bolus is from 1 to 50 ml, preferably from 5-20 ml, more preferably from 8-12 ml.

The present inventors have found that providing to dysphagic patients a bolus having an increased cohesiveness due to its extensional viscosity, as opposed to the effects of shear viscosity, dramatically reduces the amount of swallowing effort for these patients, as well as the risk of residue build-up in the oropharyngeal and/or esophageal tracts. As such, boluses having increased cohesiveness provide improved nutritional intake of dysphagic patients by enabling them to swallow a wider variety of food and beverage products safely and comfortably. This is achieved by improving bolus integrity and thus lending confidence to the patient in being able to consume the different products. The nutritional improvement achieved by an improved food and water intake may lead to an overall healthier condition of the patient and prevent further decline.

Therefore, the bolus of the present invention is not only modified with regard to its shear viscosity, but with regard to at least one further rheological property such as its cohesiveness.

Shear viscosity is a commonly measured rheological property, which is often referred to as simply viscosity, and which may be determined by any method known in the art. In the present invention, shear viscosity was determined using concentric cylinders in a standard research-grade rheometer (Anton Paar MCR). Said parameter describes the reaction of a material to applied shear stress. In other words, shear viscosity is the ratio between “stress” (force per unit area) exerted on the surface of a fluid, in the lateral or horizontal direction, to the change in velocity of the fluid as you move down in the fluid (a “velocity gradient”).

Cohesiveness is a parameter that relates to the ability of a portion of liquid to hold together when being stretched (extended, elongated) in a flow, e.g. passing through a constriction, dewetting of a drop on a surface or thinning of a liquid filament.

In the context of the present disclosure, the relaxation time of a bolus as a measure of its cohesiveness was determined by a Capillary Breakup Extensional Rheometry (CaBER) experiment.

The Capillary Breakup Extensional Rheometer is an example for a rheometer applying extensional stress. During the CaBER experiment as performed herein for measuring the relaxation time of the bolus, a drop of said bolus is placed between two vertically aligned and parallel circular metal surfaces, both having a diameter of 6 mm. The metal surfaces are then rapidly separated linearly over a time interval of 50 ms (milliseconds). The filament formed by this stretching action subsequently thins under the action of interfacial tension and the thinning process is followed quantitatively using a laser sheet measuring the filament diameter at its mid-point. The relaxation time in a CaBER experiment is determined by plotting the normalized natural logarithm of the filament diameter during the thinning process versus time and determining the slope of the linear portion (din (D/D0)/dt) of this curve, where D is the filament diameter, D0 the filament diameter at time zero and t the time of filament thinning. The relaxation time in this context is then defined as minus one third (−⅓) times the inverse of this slope, i.e. −1/(3dIn(D/D0)/dt).

Preferably, the filament diameter of the bolus decreases less than linearly, and more preferably exponentially in time during a CaBER experiment.

In one preferred embodiment, the bolus is a cohesive thin liquid having (i) a shear viscosity of less than about 50 mPas, preferably from 5 to 45 mPas, more preferably from 10 to 40 mPas, and most preferably from 20 to 30 mPas, when measured at a shear rate of 50 s−1; and (ii) a relaxation time, determined by a Capillary Breakup Extensional Rheometry (CaBER) experiment, of more than 10 ms (milliseconds) at a temperature of 20° C.

In another preferred embodiment, the bolus is a cohesive thickened liquid having (i) a shear viscosity of more than about 50 mPas, preferably from 55 to 350 mPas, more preferably from 60 to 200 mPas, and most preferably from 70 to 100 mPas, when measured at a shear rate of 50 s−1; and (ii) a relaxation time, determined by a Capillary Breakup Extensional Rheometry (CaBER) experiment, of more than 10 ms (milliseconds) at a temperature of 20° C.

It is particularly preferred that the cohesive liquid bolus of the invention has a relaxation time of less than about 2000 ms, preferably from about 20 ms to about 1000 ms, more preferably from about 50 ms to about 500 ms, and most preferably from about 100 ms to about 200 ms, at a temperature of 20° C.

A further embodiment relates to the cohesive liquid bolus in diluted form, preferably in an aqueous dilution ranging from 2:1 to 50:1, more preferably from 3:1 to 20:1 and most preferably from 5:1 to 10:1. By way of example, a dilution of 2:1 means that 1 part of nutritional product is diluted in 2 parts of water.

A further embodiment relates to the cohesive liquid bolus in administrable form, which may preferably selected from the group consisting of a pharmaceutical formulation, a nutritional formulation, a nutritional supplement, a dietary supplement, a functional food, a beverage product, a full meal, a nutritionally complete formula, and combinations thereof.

### Biopolymers

The cohesive liquid bolus of the present invention comprises an aqueous solution of at least one food grade biopolymer selected from the group consisting of botanical hydrocolloids, microbial hydrocolloids, animal hydrocolloids, algae hydrocolloids and any combination thereof.

It is preferred that these biopolymers are comprised in the cohesive liquid bolus in a concentration of from at least 0.01 wt % to 25 wt %, preferably from at least 0.1 wt % to 15 wt %, and most preferably from at least 1 wt % to 10 wt %.

Suitable algae hydrocolloids preferably include agar, carrageenan, alginate or combinations thereof. The microbial hydrocolloids may be selected from xanthan gum, gellan gum, curdlan gum, or combinations thereof.

Suitable microbial hydrocolloids preferably include xanthan gum, gellan gum, curdlan gum, or combinations thereof.

Suitable animal hydrocolloids preferably include hyaluronic acid, glucosamine sulphate, chondroitin sulphate, collagen, collagen peptides, or combinations thereof.

It is particularly preferred that the at least one food grade biopolymer is selected from botanical hydrocolloids. The latter may preferably be selected from plant-extracted gums, plant-derived mucilages, and combinations thereof.

As used herein, plant-extracted gums preferably include any one of okra gum, glucomannans (konjac mannan), galactomannans (tara gum, locust bean gum, guar gum, fenugreek gum), tamarind gum, cassia gum, gum Arabic (acacia gum), gum ghatti, pectins, modified celluloses (e.g., carboxymethyl cellulose, methyl cellulose, hydroxylpropyl methyl cellulose, hydroxypropyl cellulose), tragacanth gum, karaya gum, and combinations thereof. Okra gum is particularly preferred.

As used herein, plant-derived mucilages are preferably selected from the group consisting of kiwi fruit mucilage, cactus mucilage, chia seed mucilage, psyllium mucilage, mallow mucilage, flax seed mucilage, marshmallow mucilage, ribwort mucilage, mullein mucilage, cetraria mucilage, and combinations thereof. In a preferred embodiment, the plant-derived mucilage is kiwi fruit mucilage and/or cactus mucilage.

In the context of this disclosure, kiwi fruit mucilage is preferably derived from the stem pith of kiwi fruit, which contains about 20% of mucilage and typically represents the plant waste material remaining from kiwi fruit agriculture.

Further in this context, the gums and mucilages are preferably food grade and can be commercially obtained from numerous suppliers.

Alternatively, the above gums and mucilages may be obtained by any suitable extraction method known in the art. For example, gums and mucilages may be extracted by a method comprising the steps of soaking the raw plant material with 10 times of its weight of distilled water and keeping it overnight. A viscous solution is obtained, which is passed through a muslin cloth. The gum or mucilage is precipitated by addition of 95% by weight of ethanol in a ratio of about 1:1 by continuous stirring. A coagulated mass is obtained, which is subsequently dried in an oven at 40 to 45° C., powdered by passing through a sieve and stored in an airtight container.

In a particularly preferred embodiment, the cohesive thin liquid bolus comprises a food grade biopolymer selected from okra gum, cactus mucilage and kiwi fruit mucilage, or any combination thereof.

In another particularly preferred embodiment, the cohesive thickened liquid bolus comprises at least one food grade biopolymer selected from okra gum, cactus mucilage, kiwi fruit mucilage, and combinations thereof and a further food-grade biopolymer selected from starch, modified starch, xanthan gum, guar gum, carageenan, tara gum, locust been gum, alginates and pectins.

### Bioactive Compounds

The cohesive liquid bolus of the present invention further comprises at least one bioactive compound selected from anabolic compounds, anti-catabolic compounds, cell function or neuromuscular junction stimulating compounds and cell energy metabolism stimulating compounds.

Suitable anabolic compounds preferably include leucine, leucine metabolites, α-hydroxyisocaproic acid (HICA), glutamine, arginine, citrulline, creatine, whey, medium chain fatty acids (MCFA) and combinations thereof;

Suitable anti-catabolic compounds preferably include polyunsaturated fatty acids (PUFA), ω-3 fatty acids, carnitine, creatine, and combinations thereof;

Suitable cell function or neuromuscular junction stimulating compounds preferably include choline, vitamin D, creatine, Oleoylethanolamide (OEA), resveratrol, and combinations thereof;

Suitable cell energy metabolism stimulating compounds preferably include antioxidants, coenzyme Q10, creatine, lipoic acid, carnitine, resveratrol, medium chain fatty acids (MCFA) and combinations thereof.

As used herein, the term “antioxidant” is understood to include any one or more of various substances such as beta-carotene (a vitamin A precursor), vitamin C, vitamin E, and selenium that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include carotenoids, coenzyme Q10 (“CoQ10”), flavonoids, glutathione Goji (wolfberry), hesperidin, lactowolfberry, lignan, lutein, lycopene, polyphenols, selenium, vitamin A, vitamin B1, vitamin B6, vitamin B12, vitamin C, vitamin D, vitamin E, zeaxanthin, or combinations thereof.

Further, as used herein, medium chain fatty acids (MCFA) are understood to include any kind of fatty acid having one or more aliphatic tails of 6-12 carbon atoms, including medium-chain triglycerides.

Further, as used herein, polyunsaturated fatty acids (PUFA) are understood to include any kind of fatty acid having more than a single carbon-carbon double bond.

Further, as used herein, ω-3 fatty acids are understood to include α-linolenic acid (ALA), docosahexaenoic acid (DHA) and eicosapentaenoic acid (EPA), etc and combinations thereof.

### Rigid Particles

In a further embodiment, the bolus of the invention may comprise a suspension of rigid particles in a cohesive liquid, preferably wherein the rigid particles have a size of from 100 nm to 1 mm, preferably from 200 nm to 900 nm, from 300 nm to 800 nm, from 400 nm to 700 nm, or from 500 nm to 600 nm. It is also preferred that these rigid particles are comprised in the bolus in an amount of from 1 to 50% by volume, preferably in an amount of from 5 to 40% by volume, 10 to 30% by volume, or 15 to 20% by volume.

Moreover, it is particularly preferred that the rigid particles have an elongated shape, which means that they have an aspect ratio of larger than 1.0.

In the context of this disclosure, the term “rigid” means that the particles show no measurable deformation under the forces encountered during swallowing.

The rigid particles may be comprised of any food grade material, and are preferably selected from sucrose crystals, cocoa particles, coffee particles, mustard particles, microcrystalline cellulose particles, starch and modified starch granules, protein particles, and any combination thereof.

In the context of this disclosure, the particle size is expressed in terms of the average equivalent particle diameter. In the context of this disclosure, the equivalent particle diameter refers to the diameter of a sphere of equal volume as the particle volume, which may be determined by any suitable method known in the art. Preferably, the equivalent particle diameter is determined by laser diffraction, e.g. using a Malvern® Mastersizer instrument. Further, in this context, the average equivalent particle diameter is based on a number average, which is to be understood as the arithmetic mean of all particle diameters in a sample, usually reported as D[1,0].

In the context of this disclosure, % by volume signifies the percentage of the volume of all rigid particles in the suspension as a whole, per total volume of said suspension.

The presence of such rigid particles in the bolus of the invention was found to locally enhance extensional flow and to thereby increase extensional stresses, leading to a higher apparent extensional viscosity of said product.

### Further Potential Ingredients

The bolus may further comprise a high molecular weight protein, which is preferably selected from collagen-derived proteins such as gelatin, plant proteins such as potato, pea, lupin, etc., or other proteins of sufficiently high molecular weight (MW=100 kDa and above).

The bolus may further comprise a source of dietary protein including, but not limited to animal protein (such as meat protein or egg protein), dairy protein (such as casein, caseinates (e.g., all forms including sodium, calcium, potassium caseinates, casein hydrolysates, whey (e.g., all forms including concentrate, isolate, demineralized), whey hydrolysates, milk protein concentrate, and milk protein isolate), vegetable protein (such as soy protein, wheat protein, rice protein, and pea protein), or combinations thereof. In a preferred embodiment, the protein source is selected from the group consisting of whey, chicken, corn, caseinate, wheat, flax, soy, carob, pea, or combinations thereof.

The bolus may further comprise a source of carbohydrates. Any suitable carbohydrate may be used in the bolus of the invention including, but not limited to, sucrose, lactose, glucose, fructose, corn syrup solids, maltodextrin, modified starch, amylose starch, tapioca starch, corn starch or combinations thereof.

The bolus may further comprise a source of fat. The source of fat may include any suitable fat or fat mixture. For example, the fat source may include, but is not limited to, vegetable fat (such as olive oil, corn oil, sunflower oil, rapeseed oil, hazelnut oil, soy oil, palm oil, coconut oil, canola oil, lecithins, and the like), animal fats (such as milk fat) or combinations thereof.

The bolus may further comprise one or more prebiotics. As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The bolus may further comprise one or more probiotics. As used herein, probiotic micro-organisms (hereinafter “probiotics”) are food-grade micro-organisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. In general, it is believed that these micro-organisms inhibit or influence the growth and/or metabolism of pathogenic bacteria in the intestinal tract. The probiotics may also activate the immune function of the host. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The bolus may comprise one or more further amino acids. Non-limiting examples of suitable amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

The bolus may further comprise one or more synbiotics, sources of ω-3 fatty acids, and/or phytonutrients. As used herein, a synbiotic is a supplement that contains both a prebiotic and a probiotic as defined above that work together to improve the microflora of the intestine. Non-limiting examples of sources of ω-3 fatty acids include fish oil, krill, poultry, eggs, or other plant or nut sources such as flax seed, walnuts, almonds, algae, modified plants, etc. Non-limiting examples of phytonutrients include quercetin, curcumin and limonin and combinations thereof.

The bolus may also comprise one or more further antioxidants including carotenoids, coenzyme Q10 (“CoQ10”), flavonoids, glutathione Goji (wolfberry), hesperidin, lactowolfberry, lignan, lutein, lycopene, polyphenols, selenium, vitamin A, vitamin B1, vitamin B6, vitamin B12, vitamin C, vitamin D, vitamin E, zeaxanthin, or combinations thereof.

The bolus may further comprise fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

The bolus may further comprise other functional ingredients including chitosans and protein aggregates. Chitosans are linear polysaccharides composed of randomly distributed β-(1-4)-linked D-glucosamine (deacetylated unit) and N-acetyl-D-glucosame (acetylated unit). Among other potential benefits, chitosans have natural antibacterial properties, aid in drug delivery, and are known to rapidly clot blood. Protein aggregates are coalescences of miss-folded proteins driven by interactions between solvent-exposed hydrophobic surfaces that are normally buried within a protein's interior.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art.

Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

### Use

The cohesive liquid bolus of the invention may preferably be used in treating a swallowing disorder in a patient in need of same.

In a further embodiment, said bolus may be particularly used in supporting, maintaining and/or improving strength and/or muscle health in a patient suffering from a swallowing disorder.

In the context of the present invention, the term “swallowing disorder” refers to any kind of physiologic dysfunction and/or disorder that is associated with difficulties and/or an impairment of swallowing, and to the symptoms thereof, which in medical terms is referred to as dysphagia, including esophageal and oral pharyngeal dysphagia, and aspiration.

As used herein, the terms “treating”, “treatment” and “to treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treating” and “to treat” are also intended to include the enhancement of one or more primary prophylactic or therapeutic measures.

The terms “treatment,” “treating” and “to treat” further intended to include the dietary management of a disease or condition or the dietary management for prophylaxis or prevention a disease or condition.

As used herein, the term “patient” is understood to include a mammal such as an animal and, more preferably, a human that is receiving or intended to receive treatment, as it is herein defined. While the terms “individual” and “patient” are often used herein to refer to a human, the invention is not so limited. Accordingly, the terms “individual” and “patient” refer to any animal, mammal or human having or at risk for a medical condition that can benefit from the treatment.

In this context, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

In a further embodiment, the cohesive liquid bolus of the invention may be used in promoting safe swallowing of nutritional products, and/or for use in mitigating the risks of aspiration during swallowing of nutritional products. These methods include administering to a patient in need of same a nutritional product comprising the cohesive liquid bolus of the invention.

As used herein, the term “nutritional product” includes a nutritional formulation, a nutritional supplement, a dietary supplement, a functional food, a beverage product, a full meal, a nutritionally complete formula, and combinations thereof.

### Method

The present invention further provides a method for making a bolus selected from a cohesive liquid, the method comprising the steps of: (1) Providing an aqueous solution of at least one food grade biopolymer capable of providing to the bolus: (i) a shear viscosity of less than about 400 mPas, and (ii) a relaxation time, determined by a Capillary Breakup Extensional Rheometry (CaBER) experiment, of more than 10 ms (milliseconds) at a temperature of 20° C.; and (2) Adding to said aqueous solution at least one bioactive compound selected from: (a) Anabolic compounds; (b) Anti-catabolic compounds; (c) Cell function or neuromuscular junction stimulating compounds; and (d) Cell energy metabolism stimulating compounds.

In the inventive method, each one of the terms “food grade biopolymer”, “shear viscosity”, “relaxation time”, “anabolic compounds”, “anti-catabolic compounds”, “cell function or neuromuscular junction stimulating compounds” and “cell energy metabolism stimulating compounds” is preferably defined as set out above. Most preferably, in the inventive method the bolus selected from a cohesive liquid is the cohesive liquid bolus according to the present invention.

In a preferred embodiment, the present method comprises an optional further step of diluting the bolus, preferably in an aqueous dilution ranging from 2:1 to 50:1, more preferably from 3:1 to 20:1 and most preferably from 5:1 to 10:1.

In a further preferred embodiment, the inventive method comprises a further step of bringing the bolus in an administrable form selected from the group consisting of a pharmaceutical formulation, a nutritional formulation, a nutritional supplement, a dietary supplement, a functional food, a beverage product, a full meal, a nutritionally complete formula, and combinations thereof.

### Kit

Finally, the present invention provides a kit comprising one or more containers comprising a cohesive liquid bolus, each container comprising a cohesive thin liquid bolus or a cohesive thickened liquid bolus, respectively. Preferably, the cohesive thin liquid bolus and cohesive thickened liquid bolus are characterized as defined above.

1. A bolus selected from a cohesive liquid having
a shear viscosity of less than about 400 mPas, and
a relaxation time, determined by a Capillary Breakup Extensional Rheometry (CaBER) experiment, of more than 10 ms (milliseconds) at a temperature of 20° C.;
the bolus comprising:
an aqueous solution of at least one food grade biopolymer selected from the group consisting of botanical hydrocolloids, microbial hydrocolloids, animal hydrocolloids, algae hydrocolloids and any combination thereof;
and
at least one bioactive compound selected from the group consisting of:
anabolic compounds;
anti-catabolic compounds;
cell function or neuromuscular junction stimulating compounds; and
cell energy metabolism stimulating compounds.

2. The bolus according to claim 1, wherein
the anabolic compounds are selected from the group consisting of leucine, leucine metabolites, α-hydroxyisocaproic acid (HICA), glutamine, arginine, citrulline, creatine, whey, medium chain fatty acids (MCFA) and combinations thereof;
the anti-catabolic compounds are optionally selected from the group consisting of polyunsaturated fatty acids (PUFA), ω-3 fatty acids, carnitine, creatine, and combinations thereof;
the cell function or neuromuscular junction stimulating compounds are selected from the group consisting of choline, vitamin D, creatine, Oleoylethanolamide (OEA), resveratrol, and combinations thereof; and
the cell energy metabolism stimulating compounds are selected from the group consisting of antioxidants, coenzyme Q10, creatine, lipoic acid, carnitine, resveratrol, medium chain fatty acids (MCFA) and combinations thereof.

3. The bolus according to claim 1, wherein the bolus is selected from a cohesive thin liquid having
a shear viscosity of less than about 50 mPas, when measured at a shear rate of 50 s−1;
and
a relaxation time, determined by a Capillary Breakup Extensional Rheometry (CaBER) experiment, of more than 10 ms (milliseconds) at a temperature of 20° C.

4. The bolus according to claim 1, wherein the bolus is selected from a cohesive thickened liquid having
a shear viscosity of more than about 50 mPas, when measured at a shear rate of 50 s−1;
and
a relaxation time, determined by a Capillary Breakup Extensional Rheometry (CaBER) experiment, of more than 10 ms (milliseconds) at a temperature of 20° C.

5. The bolus according to claim 1, wherein the relaxation time is less than about 2000 ms at a temperature of 20° C.

6. The bolus according to claim 1, wherein the filament diameter of the bolus decreases less than linearly during a CaBER experiment.

7. The bolus according to claim 1, wherein the aqueous solution comprises the at least one food grade biopolymer in a concentration of from at least 0.01 wt % to 25 wt %.

8. The bolus according to claim 1, wherein the food grade biopolymer is a botanical hydrocolloid selected from plant-extracted gums, plant-derived mucilages, or combinations thereof.

9. The bolus according to claim 1 in administrable form selected from the group consisting of a pharmaceutical formulation, a nutritional formulation, a nutritional supplement, a dietary supplement, a functional food, a beverage product, a full meal, a nutritionally complete formula, and combinations thereof.

10. A method for treating a swallowing disorder, in promoting safe swallowing of nutritional products, and/or mitigating the risks of aspiration during swallowing of nutritional products in a patient in need of same comprising administering a bolus selected from a cohesive liquid having
a shear viscosity of less than about 400 mPas, and
a relaxation time, determined by a Capillary Breakup Extensional Rheometry (CaBER) experiment, of more than 10 ms (milliseconds) at a temperature of 20° C.;
the bolus comprising:
an aqueous solution of at least one food grade biopolymer selected from the group consisting of botanical hydrocolloids, microbial hydrocolloids, animal hydrocolloids, algae hydrocolloids and any combination thereof;
and
at least one bioactive compound selected from the group consisting of:
anabolic compounds;
anti-catabolic compounds;
cell function or neuromuscular junction stimulating compounds; and
cell energy metabolism stimulating compounds.

11. A method for supporting, maintaining and/or improving strength and/or muscle health in a patient suffering from a swallowing disorder comprising administering a bolus selected from a cohesive liquid having
a shear viscosity of less than about 400 mPas, and
a relaxation time, determined by a Capillary Breakup Extensional Rheometry (CaBER) experiment, of more than 10 ms (milliseconds) at a temperature of 20° C.;
the bolus comprising:
an aqueous solution of at least one food grade biopolymer selected from the group consisting of botanical hydrocolloids, microbial hydrocolloids, animal hydrocolloids, algae hydrocolloids and any combination thereof;
and
at least one bioactive compound selected from the group consisting of:
anabolic compounds;
anti-catabolic compounds;
cell function or neuromuscular junction stimulating compounds; and
cell energy metabolism stimulating compounds.

12. A method for making a bolus selected from a cohesive liquid, the method comprising the steps of:
providing an aqueous solution of at least one food grade biopolymer capable of providing to the bolus:
a shear viscosity of less than about 400 mPas, and
a relaxation time, determined by a Capillary Breakup Extensional Rheometry (CaBER) experiment, of more than 10 ms (milliseconds) at a temperature of 20° C.;
and
adding to the aqueous solution at least one bioactive compound selected from the group consisting of:
anabolic compounds;
anti-catabolic compounds;
cell function or neuromuscular junction stimulating compounds; and
cell energy metabolism stimulating compounds.

13. The method according to claim 12, wherein
the anabolic compounds are selected from the group consisting of leucine, leucine metabolites, α-hydroxyisocaproic acid (HICA), glutamine, arginine, citrulline, creatine, whey, medium chain fatty acids (MCFA) and combinations thereof;
the anti-catabolic compounds are selected from the group consisting of polyunsaturated fatty acids (PUFA), ω-3 fatty acids, carnitine, creatine, and combinations thereof;
the cell function or neuromuscular junction stimulating compounds are selected from the group consisting of choline, vitamin D, creatine, Oleoylethanolamide (OEA), resveratrol, and combinations thereof; and/or
the cell energy metabolism stimulating compounds are selected from the group consisting of antioxidants, coenzyme Q10, creatine, lipoic acid, carnitine, resveratrol, medium chain fatty acids (MCFA) and combinations thereof.

14. The method according to claim 12, wherein the bolus is selected from a cohesive thin liquid, and wherein an aqueous solution of at least one food grade biopolymer is provided which is capable of providing to the bolus:
a shear viscosity of less than about 50 mPas, when measured at a shear rate of 50 s−1, and
a relaxation time, determined by a Capillary Breakup Extensional Rheometry (CaBER) experiment, of more than 10 ms (milliseconds) at a temperature of 20° C.

15. The method according to claim 12, wherein an aqueous solution of at least one food grade biopolymer is provided which is capable of providing to the bolus:
a shear viscosity of more than about 50 mPas, when measured at a shear rate of 50 s−1, and
a relaxation time, determined by a Capillary Breakup Extensional Rheometry (CaBER) experiment, of more than 10 ms (milliseconds) at a temperature of 20° C.
